(19) United States
(12) Patent Application Publication
(10) Pub. No.: US 2011/0295243 A1
(43) Pub. Date: Dec. 1, 2011

(54) Laser-based methods and systems for corneal surgery

(76) Inventor: Gholam A. Peyman, Sun City, AZ (US)

(21) Appl. No.: 12/802,204

(22) Filed: Jun. 1, 2010

(51) Int. Cl. A61F 9/01

(52) U.S. Cl. 606/5

(57) ABSTRACT

A method for surface ablation of cornea tissue comprising the steps of (i) providing a laser source that is adapted to generate and transmit focused pulsed laser energy, the laser source including a delivery head that is adapted to direct the laser energy to a target structure of an eye, (ii) disposing the delivery head a spaced distance from the target eye structure, and (iii) transmitting the laser energy to the target eye structure, whereby the surface of the eye structure tissue is primarily, more preferably, solely ablated.

### FIELD OF THE INVENTION

The present invention relates to systems and methods for corneal and intraocular surgery. More particularly, the present invention relates to laser-based methods and systems for performing surface ablation of cornea tissue.

### BACKGROUND OF THE INVENTION

Various surgical procedures have been developed and employed to correct refractive defects (or errors) and/or treat eye diseases. Mechanical methods were initially employed to correct refractive defects by changing the curvature of the eye. These mechanical methods involve removal of a thin layer of tissue from the cornea by a microkeratome, freezing the tissue at the temperature of liquid nitrogen, and re-shaping the tissue in a specially designed lathe. The thin layer of tissue is then re-attached to the eye by suture.

As is well known in the art, there are, however, several significant drawbacks and disadvantages associated with mechanical surgical methods. Among the disadvantages are the lack of reproducibility and, hence, poor predictability of surgical results.

More recently, various laser-based methods and systems have been developed and employed to correct refractive defects and to perform general eye surgery. The laser-based methods and systems make use of the coherent radiation properties of lasers and the precision of the laser-tissue interaction.

A CO₂ laser was one of the first to be applied in this field. Peyman, et al., in Ophthalmic Surgery, vol. 11, pp. 325-9, 1980, reported laser burns of various intensity, location and pattern that were produced on rabbit corneas. Horn, et al., in the Journal of Cataract Refractive Surgery, vol. 16, pp. 611-6, 1990, also reported that a curvature change in rabbit corneas had been achieved with a Co:MgF₂ laser by applying specific treatment patterns and laser parameters.

The ability to produce burns on the cornea by either a CO₂ laser or a CO:MgF₂ laser relies on the absorption in the tissue of the thermal energy emitted by the laser. Histologic studies of the tissue adjacent to burn sites caused by a CO₂ laser have, however, revealed extensive damage characterized by a denaturalized zone of 5-10 μm deep and disorganized tissue region extending over 50 μm deep. CO₂ laser and CO:MgF₂ lasers are thus often deemed ill-suited for eye surgery.

More recently, excimer lasers have been, and continue to be, employed to correct refractive defects and to perform general eye surgery. Excimer lasers substantially reduce, and in most instances, eliminate the drawbacks and disadvantages associated with mechanical procedures and the noted CO₂ laser and CO:MgF₂ lasers.

As is well known in the art, an excimer laser comprises a gas laser, wherein inert gases, such as argon, krypton or xenon, are mixed with another reactive gas, such as fluorine or chlorine. Under an electrical discharge, a pseudo-molecule is formed. This excited dimer or exilpex soon returns to the ground state, discharging an ultraviolet light with a wavelength that depends on the composition of the inert gas.

ArF, KrF and XeF excimer lasers typically generate and transmit laser energy (in the form of a beam) having wavelengths of approximately 193 nm, 248 nm and 308 nm, respectively. The typical laser pulse duration is in the order of 10-200 ns, with a frequency in the range of approximately 100 Hz-8 kHz.

The excimer laser beam wavelength thus has enough energy to disrupt the molecular bond of organic molecules through ablation. Illustrative are the excimer laser based methods disclosed in U.S. Pat. Nos. 4,718,418 and 4,907,586.

U.S. Pat. No. 4,718,418 discloses the use of transmitted laser energy, i.e. beam, in the ultraviolet range to achieve controlled ablative photodecomposition of one or more selected regions of a cornea. According to the disclosure, the transmitted laser beam is reduced in cross-sectional area, through a combination of optical elements, to a 0.5 mm by 0.5 mm rounded-square beam spot that is scanned over a target by deflectable mirrors. To ablate a corneal tissue surface with such an arrangement, each laser pulse would thus etch out a square patch of tissue.

Further, an etch depth of 14 μm per pulse is taught for the illustrated embodiment. This etch depth could, and in all likelihood would, result in an unacceptable level of eye damage.

U.S. Pat. No. 4,907,586 discloses another technique for tissue ablation of the cornea. The noted technique comprises focusing a laser beam into a small volume of about 25-30 μm in diameter, whereby the peak beam intensity at the laser focal point could reach about 10¹² watts/cm².

It has, however, been reported that at such a peak power level tissue molecules can, and in most instances will, be “pulled” apart under the strong electric field of the transmitted laser energy (or light), which causes dielectric breakdown of the material. See, e.g., Trokel, “YAG Laser Ophthalmic Microsurgery”.

Indeed, near the threshold of the dielectric breakdown, the laser beam energy absorption characteristics of the tissue changes from highly transparent to strongly absorbent. The reaction is typically very violent, and the effects are widely variable.

Further, the amount of tissue removed is a highly non-linear function of the incident beam power. Thus, the tissue removal rate is difficult to control. Additionally, accidental exposure of the endothelium by the laser beam is a constant concern. The noted method is accordingly often not deemed optimal for cornea surface or intraocular ablation.

Even more recently, picosecond and femtosecond lasers, i.e. lasers that emit pulsed laser energy with pulse durations in the picosecond (ps) and femtosecond (fs) range, have been employed to perform eye surgery; particularly, to separate tissue structures on or in the eye. For example, femtosecond lasers are typically employed to perform flap cuts, i.e. incisions into the eye from the side in order to produce a small flap which is folded to the side, and/or creating lamellar dissection of the cornea.

Femtosecond lasers have also been employed in cataract surgery to cut the crystalline lens into many pieces prior to its removal, glaucoma filtering procedures, tunnel creation for intracorneal ring segments. It has also been reported that femtosecond lasers may potentially be employed to treat a presbyopic eye.

There are, however, several adverse side-effects that can, and in many instances will, result from focusing femtosecond; particularly, sub-femtosecond, laser energy inside tissue. As is well known in the art, sub-picosecond (e.g., <20 ps to attosecond) pulses create multi-photon ionization and plasma at their focal point. For refractive surgery, these phenomena disrupt the tissue without the undesirable thermal damage often exhibited with longer pulses (e.g., nanosecond and greater). Accordingly, femtosecond and attosecond pulses are thus typically about three and six orders of magnitude, respectively, shorter than the threshold required for tissue ablation.

When creating an incision inside cornea tissue (as the femtosecond pulses are presently used), the energy created by short leisure energy pulses couples with the lattice after each pulse passes the tissue. The avalanche ionization and multiphoton ionization produced by short pulses enhance the breakdown or incising of the tissue further. See, Miclea, et al., “Nonlinear Refractive Index Of Porcine Cornea Studied By Z-Scan And Self-Focusing During Femtosecond Laser Processing”, Optics Express, vol. 18, No. 4, pp 3700-3707 (2010); Stuart, et al., “Laser-Induced Damage in Dielectrics with Nanosecond to Sub-picosecond Pulses”, The American Physics Society, vol. 74, No. 12 pp 2248-2251 (1995); Hammer, et al., “Shielding Properties Of Laser-Induced Breakdown In Water For Pulse Durations From 5 ns To 125 fs”, Applied Optics, vol. 36, No. 22 (1997); Heisterkamp, et al., “Nonliear Side Effects Of Fs Pulses Inside Corneal Tissue During Photodisruption” Applied Physics B—Lasers and Optics, vol. 74, pp. 419-425 (2002); Mansuripur, et al., “Self-focusing in Nonlinear Optical Media”, Optics and Photonics News, April 1998; and Poudel, et al., “Nonlinear Optical Effects During Femtosecond Photodisruption”, Optical Engineering, vol. 48(11), pp 114302-1-114302-4 (2009).

Illustrative methods for performing cornea tissue ablation with pulsed laser energy having pulse durations in the picosecond and femtosecond range are set forth in U.S. Pat. No. 5,984,916 and Pub. No. 2009/0318906A1.

In U.S. Pat. No. 5,984,916, the method for performing cornea tissue ablation comprises transmitting pulsed laser energy to the cornea having the characteristics of a low ablation energy density threshold (about 0.2 to 5 μJ/(10 μm)²) and extremely short laser pulses (having a duration of about 0.01 picoseconds to about 2 picoseconds per pulse), whereby a shallow ablation depth or region (about 0.2 μm to about 5.0 μm) is provided.

In Pub. No. 2009/0318906A1, the method for performing surface ablation of cornea tissue comprises transmitting pulsed laser energy having a pulse duration in the femtosecond range and a wavelength in the range of approximately 190 nm-380 nm. The pulse repetition rate or frequency for the treatment radiation is preferably at least about 10 kHz in the invention, but, more typically in the range of approximately 100-500 kHz. For at least wavelengths in the range of approximately 340-360 nm, the pulse energy is between approximately 0.1 nJ and 5 μJ.

There are, however, several problems that can, and in many instances will, arise when employing short pulses, e.g., pulse durations in the femtosecond range, with conventional lasers to perform surgical procedures on the eye; particularly, ablation of cornea tissue. Indeed, the use of short pulses; particularly, in the femtosecond range, can potentially result in one or more undesirable nonlinear side effects, such as self-focusing, self phase modulation, white-light continuum generation, and undesirable tissue damage. These phenomena occur when the beam is focused inside the tissue, resulting in a slight mismatch between the index of refraction and optical density of the tissue that is located in the pathway of the laser beam.

It would thus be desirable to provide methods and systems for performing eye surgery that overcome the limitations of the prior art. In particular, it would be desirable to provide improved methods and systems for performing eye surgery; particularly, cornea tissue ablation, which have accurate control of tissue removal, flexibility of ablating tissue at any desired location, and with minimal risk of undesirable tissue damage.

It is therefore an object of the present invention to provide improved methods and systems for performing eye surgery; particularly, cornea tissue ablation, which have accurate control of tissue removal, flexibility of ablating tissue at any desired location, and with minimal risk of undesirable tissue damage.

It is another object of the present invention to provide methods and systems for performing cornea tissue ablation that substantially reduce or eliminate the potential non-linear side effects often encountered when employing short laser energy pulses to perform tissue ablation.

It is another object of the present invention to provide methods and systems for performing ablation of cornea tissue, wherein the entire ablation occurs on the surface of the cornea tissue.

It is another object of the present invention to provide methods and systems for performing surface ablation of cornea tissue that eliminate the need to contact the cornea with the laser delivery head.

It is another object of the present invention to provide methods and systems for performing ablation of cornea tissue that substantially reduce the risks of infection.

### SUMMARY OF THE INVENTION

The present invention is directed to methods and systems for cornea tissue ablation, wherein the delivery head of the laser source is positioned a spaced distance from the cornea and short laser pulses are employed to incrementally ablate the surface of the cornea or an exposed surface of the corneal stroma, with minimal risk of damage to the eye.

In one embodiment of the invention, the method for performing tissue ablation of an eye structure comprises the steps of: (i) providing a laser source that is adapted to generate and transmit focused laser energy, the laser source including a delivery head that is adapted to direct the laser energy to a target structure of an eye, (ii) disposing the delivery head a spaced distance from the target eye structure, and (iii) transmitting the laser energy to the target eye structure, whereby the surface of the eye structure tissue is primarily, more preferably, solely ablated.

In certain embodiments of the invention, the target eye structure comprises the cornea.

In certain embodiments of the invention, the delivery head spaced distance from the target eye structure is in the range of approximately 1 mm-10 cm.

In certain embodiments, the laser energy is transmitted in a plurality of pulses having a pulse duration in the range of approximately 0.01-20 ps.

In certain embodiments, the wavelength of the transmitted laser energy is in the range of 380-1064 nm.

In one embodiment of the invention, the system for ablation of cornea tissue comprises: (i) a laser source that is adapted to generate and transmit focused laser energy, the laser source including a delivery head that is adapted to direct the laser energy to a target structure of an eye, and (ii) laser source control means adapted to position the delivery head a spaced distance from the target eye structure, the laser source control means being further adapted to control the transmission of the laser energy to the target eye structure, whereby the laser energy is deposited primarily at the surface of eye structure and the eye structure tissue is primarily ablated at the surface thereof.

In a preferred embodiment, the eye structure tissue is solely ablated at the surface thereof.

As set forth in detail herein, the present invention provides numerous advantages compared to prior art methods and systems for performing surgical procedures on eye structures. Among the advantages are the following:
The provision of methods and systems for performing surface ablation of cornea tissue that eliminate the need to contact the cornea with the laser delivery head.
The provision of methods and systems for performing ablation of cornea tissue that provide effective ablation of cornea tissue over a broad range of wavelengths.
The provision of methods and systems for performing ablation of cornea tissue, wherein the entire ablation occurs on the surface of the cornea tissue or the exposed corneal stroma.
The provision of methods and systems for performing ablation of cornea tissue that substantially reduce the risks of infection.
The provision of methods and systems for performing ablation of cornea tissue that substantially reduce the shielding phenomenon associated with incising tissue with a laser transmission.
The provision of methods and systems for performing ablation of cornea tissue that substantially transmit and deposit laser energy primarily on the tissue surface, whereby damage to the underlying eye structures is minimized.
The provision of methods and systems for performing ablation of cornea tissue that minimize or eliminate self-focusing of the laser beam inside the cornea.
The provision of methods and systems for performing ablation of cornea tissue that minimize or eliminate the problems associated with the release of reactive ions during incising of cornea tissue.
The provision of methods and systems for performing surgical procedures on an eye structure of a patient with the patient oriented in virtually any position.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a schematic illustration of a human eye, showing the primary structures thereof;

FIGS. 2A and 2B are schematic illustrations of a laser source having the delivery head thereof positioned a spaced distance away from a human eye, in accordance with one embodiment of the invention;

FIG. 3 is a schematic illustration of a surface ablation system of the invention, showing the position of the laser source delivery head and direction of the laser beam during a myopic correction procedure, in accordance with one embodiment of the invention;

FIG. 4 is a schematic illustration of a surface ablation system of the invention, showing the position of the laser source delivery head and laser beam during a hyperopia correction procedure, in accordance with one embodiment of the invention;

FIG. 5 is a schematic illustration of a surface ablation system of the invention, showing the position of the laser source delivery head and laser beam during a LASIK® procedure, in accordance with one embodiment of the invention; and

FIG. 6 is a schematic illustration of a surface ablation system of the invention, showing the position of the laser source delivery head and laser beam during an intracorneal inlay treatment, in accordance with one embodiment of the invention.

### DETAILED DESCRIPTION OF THE PREFERRED EMBODIMENT

Before describing the present invention in detail, it is to be understood that this invention is not limited to particularly exemplified apparatus, systems, structures or methods as such may, of course, vary. Thus, although a number of apparatus, systems and methods similar or equivalent to those described herein can be used in the practice of the present invention, the preferred apparatus, systems, structures and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “a laser pulse” includes two or more such pulses and the like.

### DEFINITIONS

The term “femtosecond range”, as used herein in conjunction with a laser pulse, means and includes includes pulse lengths or durations in the 1/1000 picosecond (ps) range up to about 1-1000 femtosecond (fs).

The terms “laser energy” and “laser beam”, are used interchangeably herein and mean and include the focused energy transmitted by a laser source, such as a Ti-sapphire laser.

The terms “patient” and “subject”, as used herein, mean and include humans and animals.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the tendency of this application and all equivalents of those claims as issued.

As will readily be appreciated by one having ordinary skill in the art, the present invention substantially reduces or eliminates the disadvantages and drawbacks associated with conventional laser-based methods and systems for performing eye surgery; particularly, ablation of cornea tissue. As discussed in detail herein, short laser pulses are employed to incrementally ablate the surface of the cornea or an exposed surface of the corneal stroma, with minimal risk of damage to the eye.

The following is a brief description of the various anatomical features of the eye, which will help in the understanding of the various features of the invention:

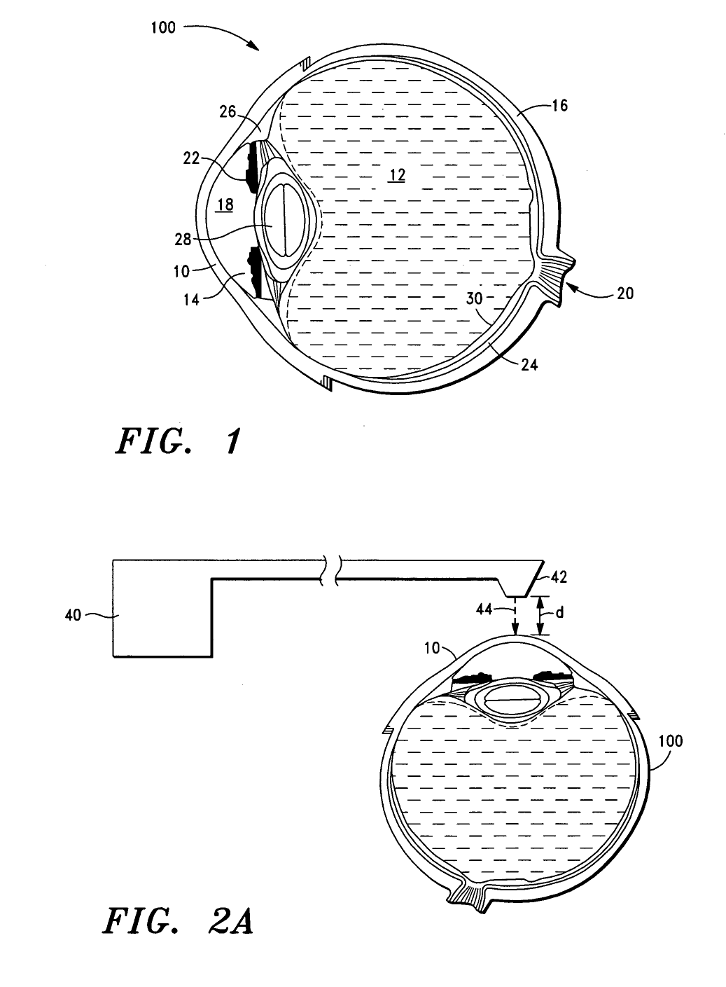

Referring to FIG. 1, the cornea 10, which is the transparent window that covers the front of the eye 100, is a lens-like structure that provides two-thirds of the focusing power of the eye. The cornea 10 is covered by an epithelium.

The cornea 10 is slightly oval, having an average diameter of about 12 mm horizontally and 11 mm vertically. The central thickness of the cornea 10 is approximately 0.5 mm and approximately 1 mm thick at the periphery.

The vitreous 12 is the largest chamber of the eye 100 (i.e. ˜4.5 ml). The vitreous 12 is a viscous transparent gel composed mostly of water. It also contains a random network of thin collagen fibers, mucopolysaccharides and hyaluronic acid.

The aqueous humor 14 occupies the anterior chamber 18 of the eye 100. The aqueous humor 14 has a volume of about 0.6 mL and provides nutrients to the cornea 10 and lens 28. The aqueous humor 14 also maintains normal lop.

The sclera 16 is the white region of the eye, i.e. posterior five sixths of the globe. It is the tough, avascular, outer fibrous layer of the eye that forms a protective envelope. The sclera is mostly composed of dense collagen fibrils that are irregular in size and arrangement (as opposed to the cornea). The extraocular muscles insert into the sclera behind the limbus.

The sclera 16 can be subdivided into 3 layers: the episclera, sclera proper and lamina fusca. The episclera is the most external layer. It is a loose connective tissue adjacent to the periorbital fat and is well vascularized.

The sclera proper, also called tenon's capsule, is the layer that gives the eye 100 its toughness. The sclera proper is avascular and composed of dense type I and III collagen.

The lamina fusca is the inner aspect of the sclera 16. It is located adjacent to the choroid and contains thin collagen fibers and pigment cells.

The pars plana is a discrete area of the sclera 16. This area is a virtually concentric ring that is located between 2 mm and 4 mm away from the cornea 10.

The mean scleral thickness±SD of the pars plana is reported to be approximately 0.53+0.14 mm at the corneoscleral limbus, significantly decreasing to 0.39±0.17 mm near the equator, and increasing to 0.9 to 1.0 mm near the optic nerve 20. At the location of the pars plana, the thickness of the sclera 16 is about 0.47±0.13 mm.

The uvea refers to the pigmented layer of the eye 100 and is made up of three distinct structures: the iris 22, ciliary body, and choroid 24. The iris 22 is the annular skirt of tissue in the anterior chamber 18 that functions as an aperture. The pupil is the central opening in the iris 22.

The ciliary body is the 6 mm portion of uvea between the iris 22 and choroid 24. The ciliary body is attached to the sclera 16 at the scleral spur. It is composed of two zones: the anterior 2 mm pars plicate, which contains the ciliary muscle 26, vessels, and processes, and the posterior 4 mm pars plana.

The ciliary muscle 26 controls accommodation (focusing) of the lens 28, while the ciliary processes suspend the lens 28 (from small fibers called zonules) and produce the aqueous humor 14 (the fluid that fills the anterior and posterior chambers and maintains intraocular pressure).

The choroid 24 is the tissue disposed between the sclera 16 and retina 30. The choroid 24 is attached to the sclera 16 at the optic nerve and scleral spur. This highly vascular tissue supplies nutrients to the retinal pigment epithelium (RPE) and outer retinal layers.

The layers of the choroid 24 (from inner to outer) include the Bruch's membrane, choriocapillaris and stroma. Bruch's membrane separates the RPE from the choroid 24 and is a permeable layer composed of the basement membrane of each, with collagen and elastic tissues in the middle.

The crystalline lens 28, located between the posterior chamber and the vitreous cavity, separates the anterior and posterior segments of the eye 100. Zonular fibers suspend the lens from the ciliary body and enable the ciliary muscle to focus the lens 28 by changing its shape.

The retina 30 is the delicate transparent light sensing inner layer of the eye 100. The retina 30 faces the vitreous and consists of two basic layers: the neural retina and retinal pigment epithelium. The neural retina is the inner layer. The retinal pigment epithelium is the outer layer that rests on Bruch's membrane and choroid 24.

Like most living organisms, eye tissue reacts to trauma, whether it is inflicted by a knife or a laser beam. One undesired reaction or side effect of incising eye tissue is the release of reactive ions within the tissue, which can, and in many instances will, initiate an inflammatory response.

Clinical studies have also shown that a certain degree of haziness develops in most eyes after surgery with conventional laser-based systems and associated techniques. The principal cause of such haziness is believed to be surface roughness resulting from cavities, grooves and ridges formed during laser etching. Clinical studies have additionally indicated that the extent of the haze depends in part on the depth of the tissue damage, which is characterized by an outer denatured layer around which is a more extended region of disorganized tissue fibers.

When an incision is created inside the cornea, a shielding phenomenon also occurs. Shielding is a caused by plasma molecules and ionization (after optical breakdown in the tissue), which results in absorption, reflection and/or scattering of subsequent laser pulses.

A gas formation is also created when such an incision is made in eye tissue. As is also well known in the art, the gas formation blocks further ablation in the area with the transmitted laser energy.

The present invention substantially reduces or eliminates the noted undesirable side effects associated with laser-based eye surgery techniques by providing methods and systems for performing ablation of cornea tissue using a laser source, wherein (i) the transmitted laser energy (or beam) has the characteristics of a low energy density threshold and short laser pulse duration(s), (ii) the delivery head of the laser source is disposed a spaced distance from the eye (i.e. a non-contact laser system), and (iii) the ablation of the cornea tissue is performed primarily, more preferably, solely on the surface of the cornea tissue.

In certain embodiments of the invention, the energy density threshold is in the range of approximately 0.01 μJ-1 mJ/(10 μm)². In certain embodiments, the energy density threshold is in the range of approximately 0.01 μJ-8 μJ/(10 μm)².

In certain embodiments, the laser pulse duration is preferably in the range of 0.01-20 ps. In certain embodiments, the laser pulse duration is preferably in the range of 1-200 fs.

In certain embodiments, the laser pulse repetition rate or frequency is preferably in the range of 10 Hz-1 MHz. In certain embodiments, the laser pulse frequency is preferably in the range of 0.1-1.0 kHz.

In certain embodiments, the wavelength of the transmitted laser radiation is preferably in the range of 380-1064 nm. In certain embodiments, the wavelength of the transmitted radiation is preferably in the range of 600-800 nm.

According to the invention, various laser sources can be employed to provide the noted laser transmission(s), including broad gain bandwidth lasers, such as Ti³:Al₂O₃, Cr:LiSrAIF₆, Nd:YLF, similar lasers, and a fiber lasers. In at least one embodiment of the invention, a Ti-sapphire laser is employed.

According to the invention, by transmitting laser energy (or a laser beam) with the Ti-sapphire laser that has a beam wavelength in the range of approximately 770-790 nm and a pulse duration in the range of approximately 145-150 femtoseconds (fs), and varying the numerical apertures of the focused lens (as is well known in the art), one can obtain an effective ablative effect on the eye surface.

According to the invention, each transmitted laser pulse is directed to a desired target structure of (or on) the eye through laser source controls means, such as described in U.S. Pat. Nos. 7,679,030, 6,716,210 and 5,280,491; which are incorporated by reference herein in their entirety.

In a preferred embodiment of the invention, the laser source control means is also adapted to provide and control the delivery head position, whereby a predetermined spaced distance of the laser source delivery head from the target eye structure can be employed.

In certain embodiments of the invention, the laser source control means is additionally adapted to provide and regulate the emitted pulse energy, e.g., duration, frequency, etc.

In certain embodiments of the invention, the laser source control means includes focusing means, such as standard or zoom lenses, to focus the laser beam on the target eye structure surface.

In certain embodiments, the laser source control means is also adapted to provide and regulate the size of the beam focal spot to, for example, keep it as small as possible to prevent the use of excessive laser energy.

In certain embodiments, the laser source control means includes a tracking system that is adapted to adjust the location of the laser beam application according to the saccadic movement of the eye.

A further key advantage of the instant invention is that the methods and systems for performing surface ablation of cornea tissue eliminate the need to contact the cornea with the laser delivery head. This is very important if the corneal surface is ablated, which produces an erosion through which germs can gain access to the corneal tissue.

As is well known in the art, the delivery head of a femtosecond laser must touch the cornea to achieve a large angle of incidence for the laser beam to focus inside the cornea. This forces the cornea to flatten to achieve a uniform stromal cut or flap to perform surgical procedures, such as forming a corneal flap in a LASIK® procedure.

Contact of the delivery head to the cornea also substantially increases the risk of infection.

The required contact of the delivery head to the cornea also contributes to the complexity of the design of the laser lens by virtue of the significant difference in the index of refraction in air versus the cornea.

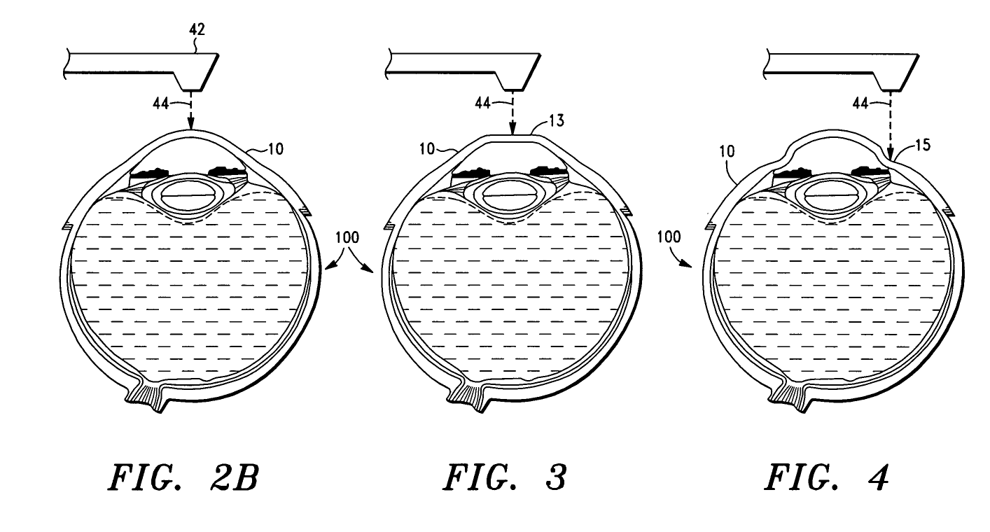

The noted issues associated with contacting the cornea with the delivery head are eliminated by virtue of the surface ablation methods and systems of the invention. As illustrated in FIGS. 2A and 2B, in a preferred embodiment of the invention, the delivery head 42 of the laser source 40 is disposed a predetermined spaced distance from the eye 100 (via the aforementioned laser source control means).

In certain embodiments of the invention, the delivery head 42 spacing, i.e. distance from the delivery head 42 to the eye 100 (denoted “d”) is in the range of approximately 1 mm-10 cm. In certain embodiments of the invention, the delivery head 42 spacing is in the range of approximately 1-5 cm.

An additional key feature of the methods and systems for performing surface ablation of cornea tissue of the invention is that the entire ablation occurs on the surface of the cornea tissue. Several significant advantages are thus realized by having a spaced delivery head, i.e. the delivery head 42 is not in contact with the cornea 10, and performing surface ablation solely on the surface of the cornea or the exposed corneal stroma.

Since the laser head 42 is not in contact with the cornea 10 and the entire ablation occurs on the surface of the tissue, the formed gas and other molecules rapidly dissipate in the air and permit the subsequent laser pulses to reach the surface of the tissue. The short time delay, i.e. laser pulse duration, of the laser transmission 44 or using a painting technique on the tissue, substantially reduces or eliminates the aforementioned shielding problem.

The noted nonlinear application of the laser transmission(s) within the tissue also depletes the pulse energy and the defocused beam beyond the focal point. It is believed that this will prevent undesired energy from being transmitted beyond the focal point and, thereby, damage occurring inside the eye.

Further, since there is a significant difference between the index of refraction of air and tissue during surface ablation, the laser beam 44 can easily be focused on the tissue surface. Thus, the entire laser energy is deposited on the tissue surface, preventing damage to the underlying structures.

The non-contact ablation systems of the invention also significantly simplify the lens design for the laser beam delivery to the ocular or corneal surface, eliminating the need for sterilization or exchanges for each surgery.

Further, the laser lens does not require a high numerical aperture. As is well known in the art, lenses with a high numerical aperture are necessary in contact systems to avoid self focusing of the laser beam inside the target tissue when performing surgical procedures requiring incisions of the eye.

To prevent the laser beam from reaching the back of the eye, shorter pulses, e.g. <300 fs pulses, have been employed, such as taught in U.S. Pat. No. 5,984,916. However, as indicated above, with conventional laser systems (i.e. contact systems) this can create the undesirable side effect of self-focusing of the beam anterior to the focal point inside the cornea.

To reduce the likelihood of self focusing of the laser beam inside the cornea (and/or absorption of the beam by the tissue), longer beam wavelengths, i.e. wavelengths in the infrared wavelength range, are typically employed with conventional contact laser systems to provide sufficient penetration of the cornea tissue.

The problem of self-focusing of the laser beam inside the cornea is, however, eliminated by the surface ablation methods and systems of the invention, wherein the entire ablation of the cornea occurs on the surface of the cornea tissue.

Further, effective ablation of cornea tissue can be realized over a much broader range of wavelengths by virtue of the surface ablation methods and systems of the invention. Indeed, according to the invention, beam wavelengths form ultraviolet to infrared and beyond can be employed to achieve effective and safe surface ablation of cornea tissue.

Further, creating an optical breakdown on the surface of the tissue requires less energy than within the tissue, by virtue of the significant difference between the index of the refraction of the air and the tissue.

Creating an incision inside the tissue of a living organism; particularly, eye tissue, is also a form of photo-disruption. An undesirable side effect of incising inside eye tissue is the release of reactive ions within the issue, which are produced by optical breakdown. The release of the reactive ions or molecules can, and in most instances will, initiate an inflammatory response and haze.

The problems associated with the release of reactive ions during incising of cornea tissue are also eliminated by the surface ablation methods and systems of the invention, since most of these molecules are removed by washing of the ocular surface during laser ablation or by the tear film.

The surface ablation methods and systems of the invention also eliminate the tissue bridging and gas bubbles phenomena that occur inside the cornea tissue when incised with a femtosecond laser.

### EXAMPLES

The following examples are provided to enable those skilled in the art to more clearly understand and practice the present invention: They should not be considered as limiting the scope of the invention, but merely as being illustrated as representative thereof.

The laser source in the following examples comprises a Ti-sapphire laser. The laser energy or beam provided by the Ti-sapphire laser has the following characteristics: a wavelength in the range of approximately 775-785 nm, a pulse duration in the range of approximately 145-155 fs, and an energy density of approximately 1.0 μJ/(10 μm)².

### Example 1

Referring to FIGS. 3 and 4, the laser delivery head 42 is initially positioned a spaced distance (d) in the range of approximately 1.0-5.0 cm over the patient's cornea 10 via the laser source control means.

The size, degree and position of the laser beam 44 is selected and controlled by the laser source control means. The desired laser beam pattern, e.g. circular, scattered, linear, etc. is also selected and controlled by the laser source control means.

The noted laser beam 44 is then directed toward the eye 100 to a target eye structure, in this example, the cornea 10 via the laser head 42 (and appropriate optics and prisms) to perform myopic correction. FIG. 3 illustrates the ablation of the cornea 10, wherein a center portion 13 is flattened via the surface ablation of the cornea 10, during the myopic correction procedure.

### Example 2

In this example, the laser delivery head 42 is similarly positioned a spaced distance (d) in the range of approximately 5-10 cm over the patient's cornea 10 via the laser source control means. The laser beam 44 is then directed to the cornea 10 via the laser head 42 to perform hyperopia correction. FIG. 4 illustrates the surface ablation of the peripheral cornea 15 during the hyperopia correction procedure.

### Example 3

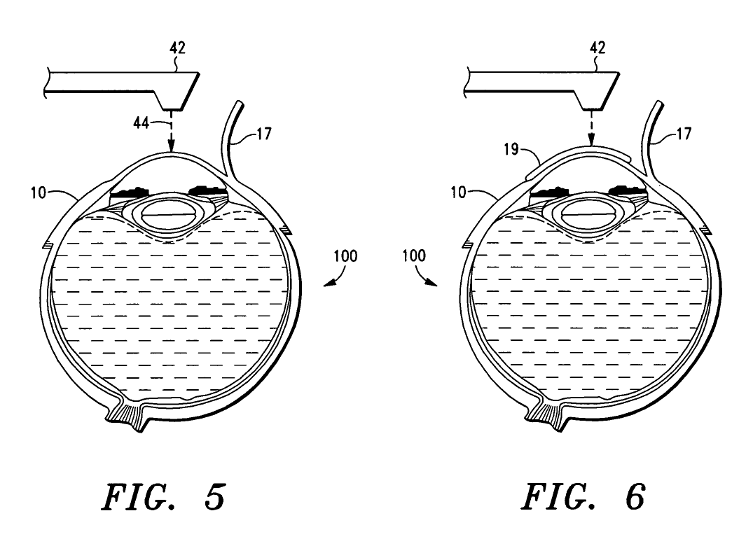

In this example, the laser delivery head 42 is similarly initially positioned a spaced distance (d) in the range of approximately 1.0-20 mm over the patient's cornea 10 via the laser source control means. The laser beam 44 is then directed to the cornea 10 via the laser head 42 to perform a LASIK® procedure, i.e. correction of a refractive error, by initially forming a corneal flap 17 and then, as illustrated in FIG. 5, performing surface ablation of the cornea 10 under the corneal flap 17.

### Example 4

In this example, the cornea has an intracorneal inlay 19 disposed therein which requires treatment.

The laser delivery head 42 is positioned a spaced distance (d) in the range of approximately 4.0-8.0 cm over the patient's cornea 10. The laser beam 44 is thereafter directed to the cornea 10 via the laser head 42 to initially form a corneal flap 17 and, thereafter, perform a corrective procedure on the inlay 19 under the corneal flap 17.

Various surgical procedures can thus be performed effectively and safely with the surface ablation methods and systems of the invention to correct refractive errors and/or to treat various eye diseases. Among the procedures are the aforementioned myopic, hyperopia, LASIK® and corneal inlay procedures, and removal of defective and/or infected tissue and tumors.

Indeed, the laser beam provided by the surface ablation methods and systems of the invention can be directed to the surface of cornea tissue to effectively and safely ablate tissue in a predetermined amount and at a predetermined location to remove defective or non-defective tissue and/or change the curvature of the cornea to achieve improved visual acuity.

As will readily be appreciated by one having ordinary skill in the art, the present invention thus provides numerous advantages compared to prior art methods and systems for performing surgical procedures on eye structures. Among the advantages are the following:
The provision of methods and systems for performing surface ablation of cornea tissue that eliminate the need to contact the cornea with the laser delivery head.
The provision of methods and systems for performing ablation of cornea tissue that provide effective ablation of cornea tissue over a broad range of wavelengths.
The provision of methods and systems for performing ablation of cornea tissue, wherein the entire ablation occurs on the surface of the cornea tissue.
The provision of methods and systems for performing ablation of cornea tissue that substantially reduce the risks of infection.
The provision of methods and systems for performing ablation of cornea tissue that substantially reduce the shielding phenomenon associated with incising tissue with a laser transmission.
The provision of methods and systems for performing ablation of cornea tissue that substantially transmit and deposit laser energy primarily on the tissue surface, whereby damage to the underlying eye structures is minimized.
The provision of methods and systems for performing ablation of cornea tissue that minimize or eliminate self-focusing of the laser beam inside the cornea.
The provision of methods and systems for performing ablation of cornea tissue that minimize or eliminate the problems associated with the release of reactive ions during incising of cornea tissue.
The provision of methods and systems for performing ablation of cornea tissue that minimize or eliminate the problems associated with variation of the pulse energy density depending on the need for doing either a myopic, hyperopic, or astigmatic surface correction using appropriate computer software.
The provision of methods and systems for performing ablation of cornea tissue that minimize or eliminate the problems associated with variation of the pulse energy created while ablating a curved surface such as the cornea depending on the need for doing either a myopic, hyperopic, or astigmatic surface correction using appropriate computer software.
The provision of methods and systems for performing surgical procedures on an eye structure of a patient with the patient's eye is stabilized with an independent vacuum system from laser head positioned on the conjunctiva and not on the cornea.
The provision of methods and systems for performing surgical procedures on an eye structure of a patient with the patient oriented in virtually any position.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

1. A method for performing incremental tissue ablation of an eye structure, comprising the steps of:
providing a femtosecond laser source that generates and transmits focused laser energy, said laser source including a delivery head that directs said laser energy to said eye structure at a spaced distance from said eye structure;
disposing said delivery head a first spaced distance in the range of approximately 1.0-5.0 cm from a surface of said eye structure;
generating first laser energy having an energy density threshold in the range of approximately 0.01 μJ-1 mJ/(10 μm)²;
transmitting said first laser energy to said eye structure to incrementally ablate said eye structure surface, said first laser energy being transmitted in the form of a beam having a first cross-sectional area with a first diameter and a first focal point, said first laser energy being transmitted in a plurality of pulses having a pulse duration in the range of 1-200 fs, a wavelength in the range of approximately 380-1064 nm, and a frequency greater than 0.1 MHz, each laser pulse having an energy density less than approximately 4 μJ/(10 μm)², all of said first laser energy being deposited on said eye structure surface and said first focal point being disposed within said eye structure surface;
controlling said first delivery head spaced distance;
controlling said first cross-sectional area of said laser energy beam;
controlling said first laser energy transmission; and
maintaining said laser beam focal point within said eye structure surface.

2. The method of claim 1, wherein said eye structure surface is solely abated.

3. The method of claim 1, wherein said eye structure comprises the cornea.

4-11. (canceled)

12. The method of claim 1, wherein said wavelength is in the range of approximately 600-800 nm.

13-14. (canceled)

15. A system for incremental tissue ablation of an eye structure, comprising:
a femtosecond laser source that generates and transmits laser energy in the form of a beam having a first cross-sectional area with a first diameter and a first focal point, said laser energy beam comprising a plurality of pulses having a pulse duration in the range of 1-200 fs, a wavelength in the range of approximately 380-1064 nm, and a frequency greater than 0.1 MHz, each laser pulse having an energy density less than approximately 4 μJ/(10 μm)²;
a delivery head that directs said laser energy to said eye structure from a first spaced distance in the range of approximately 1.0-5.0 cm from a surface of said eye structure, whereby when said laser energy is transmitted by said laser source and directed to a surface of said eye structure by said delivery head said laser beam first focal point is disposed within said eye structure surface, all of said laser energy is deposited on said eye structure surface, and said eye structure surface is incrementally ablated; and
laser source control means for positioning said delivery head said first spaced distance from said eye structure surface, and controlling said delivery head first spaced distance, controlling said pulse duration, wavelength and frequency of said laser energy, controlling said first cross-sectional area of said laser energy beam, laser source, and controlling said laser beam first focal point position with respect to said eye structure surface.

16. The system of claim 15, wherein said eye structure tissue is solely abated at the surface.

17. The system of claim 15, wherein said laser source control means includes focusing means for focusing said laser energy on said eye structure surface.

18. The system of claim 15, wherein said laser source control means includes tracking means for adjusting application of said laser energy on said eye structure in response to saccadic movement of an eye.

19-22. (canceled)
